(19) United States
(12) Patent Application Publication
(10) Pub. No.: US 2008/0026447 A1
(43) Pub. Date: Jan. 31, 2008

(54) Assay for determining factor viia inhibitor concentration in plasma samples

(75) Inventor: Yuval Blat, Wynnewood, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/788,350

(22) Filed: Apr. 19, 2007

Related U.S. Application Data
(60) Provisional application No. 60/793,473, filed on Apr. 20, 2006.

(51) Int. Cl. G01N 33/48; C12N 9/50; G01N 33/566

(52) U.S. Cl. 435/219; 436/501; 436/63

(57) ABSTRACT

This invention provides a method for determining the concentration of a factor VIIa inhibitor in a sample. A method for determining non specific binding of a factor VIIa inhibitor to proteins other than factor VIIa is also provided.

This application claims benefit to provisional application U.S. Ser. No. 60/793,473 filed Apr. 20, 2006 under 35 U.S.C. 119(e). The entire teachings of the referenced applications are incorporated herein by reference.

### FIELD OF THE INVENTION

An embodiment of the present invention relates to an assay for determining the concentration of a factor VIIa inhibitor in a plasma sample. Another embodiment of the invention relates to an assay for measuring non specific binding of factor VIIa inhibitors to plasma proteins.

### BACKGROUND OF THE INVENTION

Factor VIIa is a plasma serine protease involved in the initiation of the coagulation cascade. It binds with high affinity to tissue factor in the presence of calcium ions to form a complex with enhanced proteolytic activity (Carson, S. D. and Brozna, J. P. Blood Coag. Fibrinol. 1993, 4, 281-292). The tissue factor/factor VIIa complex initiates blood coagulation by proteolytic cleavage of factor X to factor Xa, factor IX to factor IXa and additional factor VII to factor VIIa. Ultimately, the activity of factor VIIa induces the conversion of prothrombin to thrombin. Thrombin converts fibrinogen to fibrin which forms a clot through polymerization.

While blood coagulation is essential to the regulation of an organism's hemostasis, it is also involved in many pathological conditions. For instance, thrombosis, or formation of a clot which obstructs circulation, plays a role in unstable angina, myocardial infarction, ischemic stroke, deep vein thrombosis, peripheral occlusive arterial disease, pulmonary embolism, and other diseases.

Since factor VIIa is involved in the initiation of the coagulation cascade, thrombotic disease can be treated or prevented by inhibiting factor VIIa (Girard, T. J. and Nicholson, N. S., Curr. Opin. Pharmacol. 2001, 1, 159-163). Factor VIIa inhibitors are useful anticoagulants and antithrombotic agents. Factor VIIa inhibitors useful for inhibiting the coagulation cascade have been identified. For example, U.S. Pat. No. 5,866,542 describes recombinant nematode anticoagulant proteins which inhibit factor VIIa. U.S. Pat. No. 5,843,442 discloses monoclonal antibodies or antibody fragments possessing factor VIIa inhibitory activity, and U.S. Pat. No. 5,023,236 presents tripeptides and tripeptide derivatives that inhibit factor VIIa.

It is desirable to find new compounds with improved pharmacological characteristics compared with known factor VIIa inhibitors. For example, it is preferred to find new compounds with improved factor VIIa inhibitory activity, oral bioavailability and reduced binding to plasma proteins other than factor VIIa.

Improved methods for determining the concentration of a factor VIIa (FVIIa) inhibitor in a sample are needed, particularly, methods for measuring the concentration of factor VIIa inhibitors in clinical samples. Such methods will help in selecting the appropriate dosing regime and in defining the relationships between the level of factor VIIa inhibition and therapeutic benefit or bleeding. Other coagulation factors become activated when factor VII is activated. The presence of other coagulation factors such as, for example, factor Xa can interfere with determination of factor VIIa activity and the inhibitory activity of a factor VIIa inhibitor in a sample. One reason for such interference is the lack of specificity for factor VIIa of the currently available peptide substrates which are also substrates for factor Xa and thrombin. Assays which directly measure the amidolytic activity of factor VIIa are more advantageous than assays that indirectly determine the activity of factor VIIa by detecting the activation of factor X. However, conventional assays for directly measuring factor VIIa or factor VIIa inhibitors in a sample require processing steps to separate factor VIIa and/or the factor VIIa inhibitor from other plasma proteins. A need exists for methods of determining factor VIIa activity, identifying factor VIIa inhibitors and determining the concentration of factor VIIa inhibitors that do not require separation of factor X, factor Xa and/or thrombin from the sample.

### SUMMARY OF THE INVENTION

An embodiment of the present invention provides an improved assay useful for determining the concentration of a factor VIIa inhibitor in a sample without the need to extract or separate the factor VIIa inhibitor or plasma coagulation factors from the sample. The methods of the present invention utilize the amidolytic activity of factor VIIa as a marker for factor VIIa inhibitor activity. The method involves inhibition of factor Xa activity thereby avoiding the activation of other plasma coagulation factors, such as prothrombin, that can interfere with measurement of factor VIIa activity because the peptide substrate is also hydrolyzed by these other downstream proteases. Therefore, the methods of the present invention provide increased throughput and accuracy.

Other embodiments of the present invention provide methods for determining the amount of a factor VIIa inhibitor bound to non-specific plasma proteins, i.e., proteins other than factor VIIa. The amount of a factor VIIa inhibitor bound to non-specific proteins is determined by conducting the assay for measuring the concentration of factor VIIa inhibitors in the presence and absence of plasma and comparing the activity of factor VIIa inhibitors in the presence and absence of plasma. Some embodiments provide methods for identifying factor VIIa inhibitors that have low non specific plasma protein binding, i.e., small effect of plasma on inhibitor activity.

### BRIEF DESCRIPTION OF THE FIGURES

FIG. 1A is a chart showing the chemical structures of 1-(3-(aminomethyl)phenyl)-N-(3-fluoro-2′-(methylsulfonyl)biphenyl-4-yl)-3-(trifluoromethyl)-1H-pyrazole-5-carboxamide, 2-(1-aminoisoquinolin-6-ylamino)-2-(3-ethoxy-4-isopropoxyphenyl)-N-(4-hydroxyphenylsulfonyl)acetamide, (R)-N-(5-acetamido-2-(ethylsulfonyl)benzyl)-2-(1-aminoisoquinolin-6-ylamino)-2-(3,4-dimethoxyphenyl)acetamide, (R)-2-(1-aminoisoquinolin-6-ylamino)-2-(3-ethoxy-4-isopropoxyphenyl)-N-(phenylsulfonyl)acetamide, (S)-N-(4-carbamimidoylbenzyl)-1-chloro-3-(cyclobutylamino)-8,8-diethyl-4-oxo-4,6,7,8-tetrahydropyrrolo[1,2-a]pyrazine-6-carboxamide, and 2-(5-carbamimidoyl-2-((3,5-dimethoxy-4-methylbenzamido)methyl)phenylamino)acetic acid.

FIG. 1B is a chart showing the chemical structure of 2′-((6R,6aR,11bR)-2-Carbamimidoyl-6,6a,7,11b-tetrahydro-56H-indeno[2,1-c]quinolin-6-yl)-5′-hydroxy-4′-methoxybiphenyl-4-carboxylic acid, 1-(4-methoxyphenyl)-7-oxo-6-[4-(2-oxo-1-piperidinyl)phenyl]-4,5,6,7-tetrahydro-1H-pyrazolo[3,4-c]pyridine-3-carboxamide, 1-(3-chlorophenyl)-7-oxo-6-[4-(2-oxo-1(2H)pyridinyl)phenyl]-4,5,6,7-tetrahydro-1H-pyrazolo[3 ,4-c]pyridine-3-carboxamide, 3-(1-Hydroxy-1-methyl-ethyl)-1-(4-methoxy-phenyl)-6-[4-(2-oxo-2H-pyridin-1-yl)-phenyl]-1,4,5,6-tetrahydro-pyrazolo[3,4-c]pyridin-7-one and 1-(3′-aminobenzisoxazol-5′-yl)-3-trifluoromethyl-5-[[4-[(2′-dimethylaminomethyl)imidazol-1-yl]-2-fluorophenyl]aminocarbonyl]pyrazole.

FIG. 2 is a graph showing hydrolysis of substrate D-Ile-Pro-Arg-AFC by factor VIIa in the presence of normal plasma (triangles), factor X deficient plasma (circles), and factor X deficient plasma containing a factor VIIa inhibitor (squares).

FIG. 3 is a graph showing a decrease in hydrolysis of the substrate D-Ile-Pro-Arg-AFC in plasma in the presence of factor Xa inhibitor 1-(3-(aminomethyl)phenyl)-N-(3-fluoro-2′-(methylsulfonyl)biphenyl-4-yl)-3-(trifluoromethyl)-1H-pyrazole-5-carboxamide (compound A—shaded circles) demonstrating that the factor Xa inhibitor reduces activation of the coagulation cascade. Open circles illustrate a reaction in which no inhibitor was present.

FIG. 4 is a hypothetical illustration of the effect of non specific plasma protein binding on inhibitor potency in plasma (triangles) versus buffer (squares).

FIG. 5 is a graph illustrating decreased potency of factor VIIa inhibitor, 2-(1-aminoisoquinolin-6-ylamino)-2-(3-ethoxy-4-isopropoxyphenyl)-N-(4-hydroxyphenylsulfonyl)acetamide (Compound B), in the presence of factor X deficient plasma (triangles) versus buffer (squares) thereby enabling determination of non specific protein binding: fraction bound=1-(buffer IC50/plasma IC50).

FIG. 6A is a graph showing a standard curve of a factor VIIa inhibitor (R)-N-(5-acetamido-2-(ethylsulfonyl)benzyl)-2-(1-aminoisoquinolin-6-ylamino)-2-(3,4-dimethoxyphenyl)acetamide (Compound C) concentration response in an assay containing rabbit plasma (10%), factor Xa inhibitor 1-(3-(aminomethyl)phenyl)-N-(3-fluoro-2′-(methylsulfonyl)biphenyl-4-yl)-3-trifluoromethyl)-1H-pyrazole-5-carboxamide, factor VIIa substrate D-Ile-Pro-Arg-AFC, factor VIIa, soluble tissue factor, and CaCl₂.

FIG. 6B is a graph showing inhibition of factor VIIa hydrolytic activity by sample of plasma from a rabbit dosed with (R)-N-(5-acetamido-2-(ethylsulfonyl)benzyl)-2-(1-aminoisoquinolin-6-ylamino)-2-(3,4-dimethoxyphenyl)acetamide as a function of sample dilution (sample B6). The concentration of inhibitor in the original sample is equal to: inhibitor IC50/sample dilution that gives 50% inhibition.

FIG. 7 is a graph comparing factor VIIa inhibitor (R)-N-(5-acetamido-2-(ethylsulfonyl)benzyl)-2-(1-aminoisoquinolin-6-ylamino)-2-(3,4-dimethoxyphenyl)acetamide (Compound C) concentration determination by the functional assay disclosed here and by liquid chromatography-mass spectrometry (LC-MS). A total of 22 independent samples were analyzed. The high correlation between the two methods demonstrates the reliability of the disclosed method.

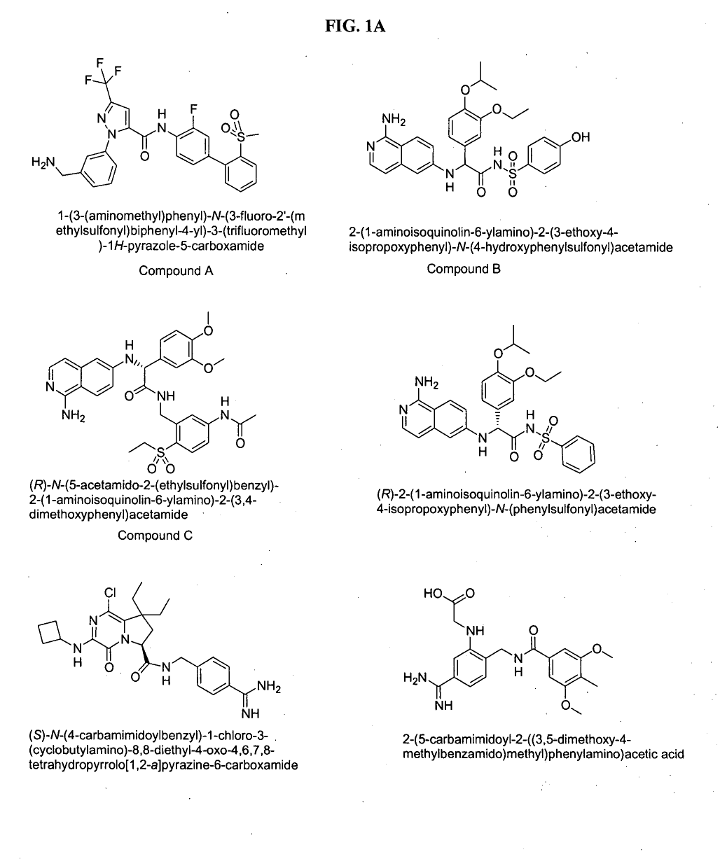

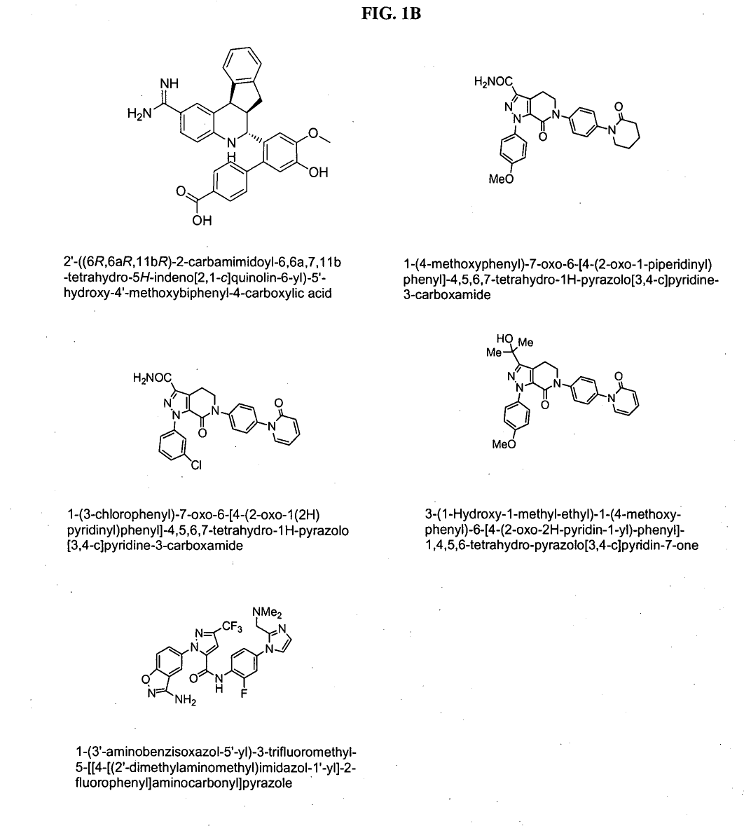

### DETAILED DESCRIPTION OF THE INVENTION

In accordance with embodiments of the present invention, other plasma coagulation factors are physically or functionally eliminated from the test sample to inhibit activity of plasma coagulation factors other than factor VIIa. More particularly, in some embodiments of the factor VIIa assays of the present invention, factor X is absent or factor Xa is inhibited. In one embodiment of the present invention, the fraction of free factor VIIa inhibitor in plasma is determined by combining factor X deficient plasma with factor VIIa, tissue factor (TF), a factor VIIa substrate and calcium. In another embodiment, the concentration of a factor VIIa inhibitor in a normal plasma sample is determined by combining it with factor VIIa, tissue factor, a factor VIIa substrate, calcium and a factor Xa inhibitor.

In one aspect of the invention the concentration of a factor VIIa inhibitor in a test plasma sample is determined by comparing the test plasma sample to a reference plasma sample having a known concentration of the factor VIIa inhibitor. An embodiment of the present invention provides a method for determining the concentration of a factor VIIa inhibitor in a test plasma sample comprising the steps of:

a) combining the test plasma sample with a solution comprising tissue factor, factor VIIa, a factor Xa inhibitor, a factor VIIa substrate, calcium, and a buffer;

b) determining the concentration of the factor VIIa inhibitor in the test plasma sample by detecting the amount of the product formed by hydrolysis of the factor VIIa substrate; and

c) comparing the amount of hydrolysis of the factor VIIa substrate in the test plasma sample to the amount of hydrolysis of a substrate in a reference plasma sample having a known concentration of the tested inhibitor to determine the amount of factor VIIa inhibitor in the test plasma sample. The amount of hydrolysis of the factor VIIa substrate may be detected by any method. Exemplary detection methods are uv or fluorescence spectrophotometry.

Examples of factor VIIa inhibitors suitable for use in the assays of the present invention are 2′-((6R,6aR, 11bR)-2-Carbamimidoyl-6,6a,7,11b-tetrahydro-56H-indeno[2,1-c]quinolin-6-yl)-5′-hydroxy-4′-methoxybiphenyl-4-carboxylic acid, (S)-N-(4-carbamimidoylbenzyl)-1-chloro-3-(cyclobutylamino)-8,8-diethyl-4-oxo-4,6,7,8-tetrahydropyrrolo[1,2-a]pyrazine-6-carboxamide (R)-2-(1-aminoisoquinolin-6-ylamino)-2-(3-ethoxy-4-isopropoxyphenyl)-N-(phenylsulfonyl)acetamide, (R)-N-(5-acetamido-2-(ethylsulfonyl)benzyl)-2-( 1-aminoisoquinolin-6-ylamino)-2-(3,4-dimethoxyphenyl)acetamide, 2-(1-aminoisoquinolin-6-ylamino)-2-(3-ethoxy-4-isopropoxyphenyl)-N-(4-hydroxyphenylsulfonyl)acetamide and 2-(5-carbamimidoyl-2-((3,5-dimethoxy-4-methylbenzamido)methyl)phenylamino)acetic acid.

Another embodiment of the present invention provides a method for determining the concentration of free factor VIIa inhibitor in a factor X deficient plasma sample comprising the steps of:

a) combining a factor X deficient test plasma sample with tissue factor, factor VIIa, a factor VIIa substrate, a factor VIIa inhibitor, calcium and a buffer;

b) determining the concentration of the free factor VIIa inhibitor in the factor X deficient test plasma sample by detecting the amount of product formed by the hydrolysis of the factor VIIa substrate; and

c) comparing the amount of hydrolysis product formed in the factor X deficient test plasma sample to an amount of product formed in a reference experiment containing no plasma and having a known concentration of the tested inhibitor. In this setting the fraction of free factor VIIa inhibitor equals: the concentration of inhibitor needed for 50% inhibition of product formation in buffer/the concentration of inhibitor needed for 50% inhibition of product formation in plasma.

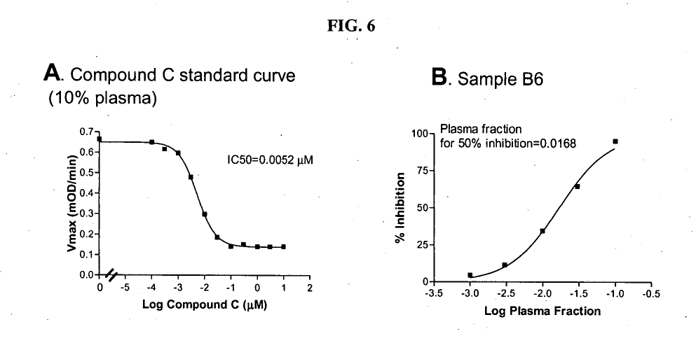

Preferably, the plasma samples are obtained from patients or animals dosed with a factor VIIa inhibitor. In some aspects, factor X deficient plasma is used. Examples of factor X deficient plasma that may be used are human congenitally deficient plasma and factor X immuno-depleted plasma. The source of calcium can be any source of calcium ions such as CaCl₂. The inhibitory activity of the factor VIIa inhibitor in the test sample is determined by measuring the detectable signal of the product formed by hydrolysis of the factor VIIa substrate and comparing the factor VIIa activity to a reference sample having a known concentration of the factor VIIa inhibitor. Preferably, as shown in FIG. 6, the activity of the test sample is compared to the activity of a standard curve determined with reference plasma having known concentrations of the test factor VIIa inhibitors. The concentration of factor VIIa inhibitor in the test sample is then calculated as follows:
Inhibitor concentration=inhibitor IC50 X plasma dilution factor resulting in 50% inhibition.

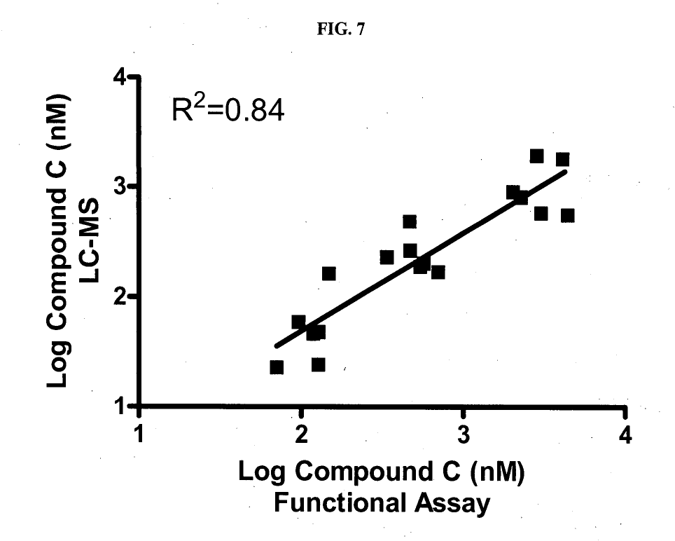

Inhibitor concentration measured using the disclosed method is accurate and reproducible. A comparison of the inhibitor concentration determined by the disclosed assay to the concentration of inhibitor in the same samples by liquid chromatography-mass spectrometry (LC-MS) shows excellent correlation as shown in FIG. 7.

In accordance with the present invention, activation of the coagulation cascade is prevented to ensure that the measured amidolytic activity originates solely from factor VIIa in a plasma sample. In accordance with the present invention, factor Xa activity can be eliminated by utilizing a factor Xa inhibitor or plasma deficient in factor X. The factor X deficient plasma can be obtained from a mammal that is congenitally deficient in factor X, such as congenitally deficient factor X human plasma (available from George King Bio-Medical, Inc., New York, N.Y.).

Examples of factor Xa inhibitors suitable for use with the present invention are 1-(3-(aminomethyl)phenyl)-N-(3-fluoro-2′-(methylsulfonyl)biphenyl-4-yl)-3-(trifluoromethyl)-1H-pyrazole-5-carboxamide, tick anticoagulant peptide (TAP), antistasin (from the Mexican leech Haementeria officinalis), 1-(4-methoxyphenyl)-7-oxo-6-[4-(2-oxo-1-piperidinyl)phenyl]-4,5,6,7-tetrahydro-1H-pyrazolo[3,4-c]pyridine-3-carboxamide, 1-(3-chlorophenyl)-7-oxo-6-[4-(2-oxo-1(2H)pyridinyl)phenyl]-4,5,6,7-tetrahydro-1H-pyrazolo[3,4-c]pyridine-3-carboxamide, 3-(1-Hydroxy-1-methyl-ethyl)-1-(4-methoxy-phenyl)-6-[4-(2-oxo-2H-pyridin-1-yl)-phenyl]-1,4,5,6-tetrahydro-pyrazolo[3,4-c]pyridin-7-one and 1-(3′-aminobenzisoxazol-5′-yl)-3-trifluoromethyl-5-[[4-[(2′-dimethylaminomethyl)imidazol-1′-yl]-2-fluorophenyl]aminocarbonyl]pyrazole.

In still another aspect, the present invention provides a method for identifying compounds that inhibit factor VIIa activity in plasma. In accordance with the present invention, factor VIIa inhibitors can be identified by combining a test compound with plasma, factor VIIa, a factor VIIa substrate, a factor Xa inhibitor, tissue factor, and calcium and comparing the factor VIIa activity in the test sample to a solution lacking the test compound. Alternatively, the factor VIIa inhibitor can be identified by combining a test factor VIIa inhibitor compound with plasma, factor VIIa, a factor VIIa substrate, a factor Xa inhibitor, tissue factor, and calcium and comparing the factor VIIa activity to the factor VIIa activity of a reference sample containing a reference factor VIIa inhibitor compound in place of the test factor VIIa inhibitor compound. Some embodiments of the present invention provide methods for identifying factor VIIa inhibitors for treating thromboembolic disorders such as arterial cardiovascular thromboembolic disorders, venous cardiovascular thromboembolic disorders, arterial cerebrovascular thromboembolic disorders, and venous cerebrovascular thromboembolic disorders.

More particularly, some of the embodiments of the present invention provide methods for identifying and monitoring factor VIIa inhibitor compounds for treating thromboembolic disorders including unstable angina, first myocardial infarction, recurrent myocardial infarction, ischemic sudden death, transient ischemic attack, stroke, atherosclerosis, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, and (f) other procedures in which blood is exposed to an artificial surface that promotes thrombosis.

In another embodiment, the present invention provides methods for identifying compounds for treating inflammatory disorders. In accordance with the present invention, compounds for treating inflammatory disorders, sepsis, acute respiratory distress syndrome, and systemic inflammatory response syndrome can be identified and monitored.

Some embodiments of the present invention provide a method for determining whether a test compound inhibits factor VIIa activity. The method comprises the steps of:

a) providing a sample comprising factor X deficient plasma, factor VIIa, a factor VIIa substrate, tissue factor and calcium;

b) determining the activity of factor VIIa in the sample in the absence of a test compound;

c) determining the activity of factor VIIa in the sample in the presence of the test compound; and

d) comparing the activity of factor VIIa in the presence and absence of the test compound wherein a decrease in activity of factor VIIa in the sample in the presence of the test compound indicates that the test compound inhibits factor VIIa activity.

Alternatively, the method for determining whether a test compound inhibits factor VIIa activity comprises the steps of:

a) providing a sample comprising plasma, factor Xa inhibitor, factor VIIa, a factor VIIa substrate, tissue factor and calcium;

b) determining the activity of factor VIIa in the sample in the absence of a test compound;

c) determining the activity of factor VIIa in the sample in the presence of the test compound; and

d) comparing the activity of factor VIIa in the presence and absence of the test compound wherein a decrease in activity of factor VIIa in the sample in the presence of the test compound indicates that the test compound inhibits factor VIIa activity.

In one embodiment of the present invention, decreased substrate hydrolyzed relative to the test compound indicates that the test compound inhibits factor VIIa.

In another aspect of the invention a patient is monitored over a period of time to identify the concentration of a factor VIIa inhibitor in the patient's plasma during the time period. For example, a patient can be monitored for 24 hours to identify the concentration of a factor VIIa inhibitor in the patient's plasma during that time period.

The assay of the present invention can be used to determine the concentration of factor VIIa inhibitor in mammalian plasma, cell extracts and tissue extracts. Moreover, the present invention also provides in vitro assays for determining the activity of factor VIIa in the presence of a factor VIIa inhibitor.

In another embodiment, the present invention provides a method for monitoring compounds for treating thromboembolic disorders that does not require separation or extraction of plasma coagulation factors. The method for monitoring compounds for treating thromboembolic disorders comprising administering to a patient in need of such treatment a therapeutically effective amount of at least one factor VIIa inhibitor compound or a pharmaceutically acceptable salt, solvate, or prodrug form thereof, obtaining a sample from the patient, and comparing the amount of inhibitor in the patient sample with a sample having a known amount of the factor VIIa inhibitor.

Some embodiments of the present invention also provide articles of manufacture, such as kits and packages for determining the concentration of factor VIIa inhibitor in a sample.

In one embodiment, the present invention provides a kit including factor VIIa, a factor VIIa substrate, a factor Xa inhibitor, tissue factor, and calcium for use in measuring or monitoring the activity or concentration of a factor VIIa inhibitor in a sample. In accordance with another embodiment, the present invention provides a kit for identifying factor VIIa inhibitors. Preferably, the kit includes at least one factor VIIa inhibitor reference compound, factor VIIa, a factor VIIa substrate, a factor Xa inhibitor, tissue factor and calcium. In an alternative embodiment, the kit for identifying factor VIIa inhibitors includes at least one factor VIIa reference compound, factor VIIa, a factor VIIa substrate, factor X deficient plasma, tissue factor and calcium.

Suitable factor VIIa substrates for use with the assay of the present invention are substrates that provide a detectable signal in response to factor VIIa activity such as for example, fluorogenic or chromogenic substrates. Substrates suitable for use in the assays of the present invention include a polypeptide portion and a detectable portion. Examples of suitable compounds that can be used to label the factor VIIa substrate are biotin, fluorescein, rhodamine and coumarin.

Preferred chromogenic substrates have a p-nitroaniline (pNA) group. A preferred chromogenic substrate is H-D-Ile-Pro-Arg-para-nitroaniline (also known as S-2288, available from Chromogenix, Orangeburg, New York). Preferred fluorogenic substrates for use in accordance with the present invention contain 6-amino-1-naphthalenesulfonamide. Other preferred fluorogenic substrates are D-Ile-Pro-Arg-7-amino-4-trifluoromethylcoumarin (D-Ile-Pro-Arg-AFC) and D-Ile-Pro-Arg-AFC*2 TFA.

The tissue factor or thromboplastin can be a partially purified preparation or a purified recombinant mammalian tissue factor. A preferred tissue factor for use in accordance with the present invention is a fragment of soluble human tissue factor having the amino acid sequence of SEQ ID NO: 1. This fragment contains amino acids 37-242 of the native sequence.

Conventional buffers used in plasma assays are suitable for use in the assay of the present invention such as a buffer including HEPES, polyethylene glycol, NaCl and bovine serum albumin.

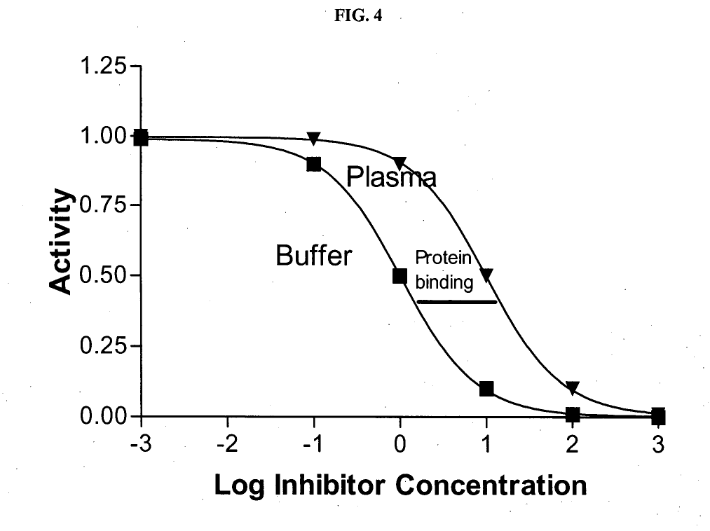

Some embodiments of the present invention provide methods for determining the plasma protein binding affinity of a factor VIIa inhibitor for other plasma proteins. Factor VIIa inhibitors having low plasma protein binding affinity for plasma proteins other than factor VIIa are preferred because more unbound factor VIIa inhibitor is available to bind and inhibit factor VIIa. The term “low protein binding factor VIIa inhibitor” as used herein means, a factor VIIa inhibitor that decreases the activity of factor VIIa in the presence or absence of other plasma proteins. Some embodiments of the present invention provide a method for determining the plasma protein binding of a factor VIIa inhibitor by incubating the test inhibitor in a first solution comprising factor X deficient plasma, tissue factor, factor VIIa, a factor VIIa substrate, such as, for example, D-Ile-Pro-Arg-AFC, calcium and a buffer, and incubating the test inhibitor in a second solution comprising tissue factor, factor VIIa and a factor VIIa substrate and a buffer without plasma. As shown in FIG. 4, the shift in inhibitor potency in the presence of the plasma is proportional to the level of plasma protein binding. The fraction of inhibitor bound to plasma protein is calculated by 1-IC50 in buffer/IC50 in plasma.

The methods of the present invention can be performed manually or performed using an automated system to achieve high-throughput. Techniques for performing high-throughput assays include use of microtiter plates or pico-, nano- or micro-liter arrays. The assays of the invention are designed to permit high throughput screening of large compound libraries, e.g., by automating the assay steps and providing candidate factor VIIa inhibitors from any source to assay. Assays which are run in parallel in high throughput format (e.g., microtiter formats on microtiter plates in robotic assays) are well known. Automated systems and methods for detecting and measuring changes in optical detection (or signal) are known. Furthermore, in the assays of the invention, it is desirable to run positive controls to ensure that the components of the assays are working properly.

It will also be appreciated that test factor VIIa inhibitors can be combined with other compounds to treat thromboembolic disorders. Examples of compounds that can be combined with factor VIIa inhibitors are one or more of potassium channel openers, calcium channel blockers, sodium hydrogen exchanger inhibitors, antiarrhythmic agents, antiatherosclerotic agents, anticoagulants, antithrombotic agents, prothrombolytic agents, fibrinogen antagonists, diuretics, antihypertensive agents, ATPase inhibitors, mineralocorticoid receptor antagonists, phospodiesterase inhibitors, antidiabetic agents, anti-inflammatory agents, antioxidants, angiogenesis modulators, antiosteoporosis agents, hormone replacement therapies, hormone receptor modulators, oral contraceptives, antiobesity agents, antidepressants, antianxiety agents, antipsychotic agents, antiproliferative agents, antitumor agents, antiulcer and gastroesophageal reflux disease agents, growth hormone agents and/or growth hormone secretagogues, thyroid mimetics, anti-infective agents, antiviral agents, antibacterial agents, antifungal agents, cholesterol/lipid lowering agents and lipid profile therapies, and agents that mimic ischemic preconditioning and/or myocardial stunning. In a preferred embodiment, the present invention provides a pharmaceutical composition wherein the at least one additional therapeutic agent is an antihypertensive agent such as ACE inhibitors, AT-1 receptor antagonists, ET receptor antagonists, dual ET/AII receptor antagonists, and vasopepsidase inhibitors, an antiarrythmic agent such as, for example, IKur inhibitors, an anticoagulant agent selected from thrombin inhibitors, other factor VIIa inhibitors, other plasma kallikrein inhibitors, factor IXa inhibitors, factor Xa inhibitors, and factor XIa inhibitors, or an antiplatelet agent selected from GPIIb/IIIa blockers, P2Y₁ and P2Y₁₂ antagonists, thromboxane receptor antagonists, and aspirin.

### EXAMPLES

The invention is illustrated by the following examples. These examples are illustrative only and do not limit the scope of the invention in any way.

### Example 1

### Reduction in Activation of the Coagulation Cascade in Factor X Deficient Plasma

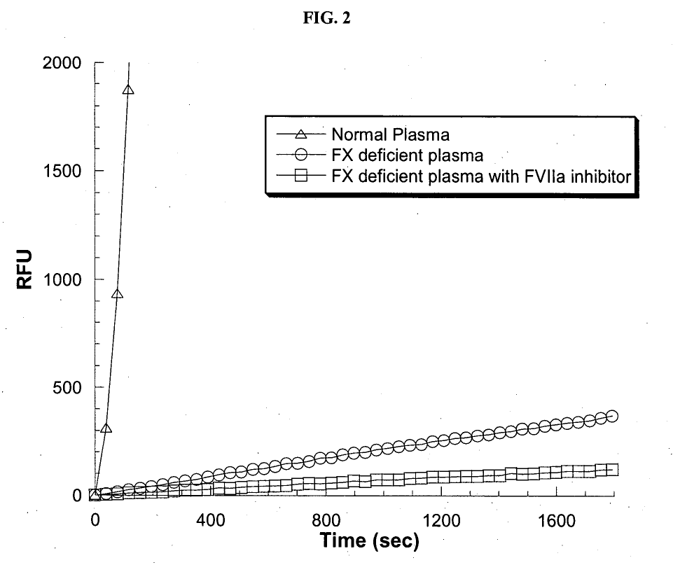

To demonstrate reduction in the activation of the coagulation cascade affects the amount of factor VIIa substrate hydrolysis, factor VIIa substrate hydrolysis was determined in the presence of plasma containing factor X and in the absence of factor X using plasma derived from a donor congenitally deficient in factor X. 85% factor X deficient plasma, 0.5 nM factor VIIa, 28 nM soluble tissue factor, 10 mM CaCl₂, and 100 nM D-Ile-Pro-Arg-AFC were diluted in 50 mM HEPES pH=7.5, 150 mM NaCl, 0.1% polyethyleneglycol 8000 and 0.1% bovine serum albumin. factor VIIa substrate hydrolysis was also determined after adding 1 μM factor VIIa inhibitor 2′-((6R,6aR,11bR)-2-carbamimidoyl-6,6a,7,11b-tetrahydro-5H-indeno[2,1-c]quinolin-6-yl)-5′-hydroxy-4′-methoxybiphenyl-4-carboxylic acid to the factor X deficient plasma sample. The assay was carried out in a fluorescence plate reader at room temperature. factor VIIa substrate hydrolysis (amidolytic activity on the substrate) was monitored for 30 minutes at an excitation wavelength of 400 nm and an emission wavelength of 535 nm. As shown in FIG. 2, plasma activation following the addition of factor VIIa, tissue factor and calcium, does not occur in factor X deficient plasma (compare circles to triangles), thus enabling the use of externally added factor VIIa as a marker for inhibitor activity without interference by other coagulation proteases. Such interference is primarily due to hydrolysis of the relatively nonspecific peptide substrate by downstream proteases such as factor Xa and thrombin. Most of the amidolytic activity in factor X deficient plasma can be attributed to factor VIIa as most of it is inhibited by the addition of the specific factor VIIa inhibitor 2′-((6R,6aR,11bR)-2-carbamimidoyl-6,6a,7,11b-tetrahydro-5H-indeno[2,1-c]quinolin-6-yl)-5′-hydroxy-4′-methoxybiphenyl-4-carboxylic acid (compare circles to squares).

### Example 2

### Reduction in Activation of the Coagulation Cascade with Factor X Inhibitor

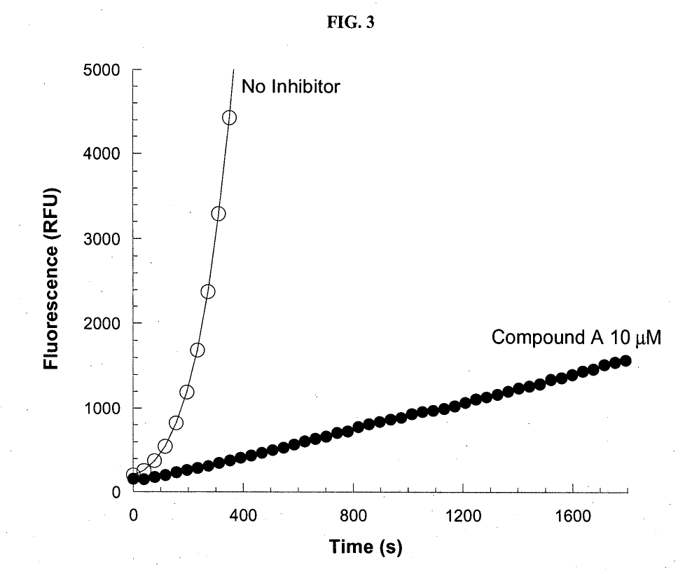

A first sample containing 11% rabbit plasma, 2.5 nM factor VIIa, 28 nM soluble tissue factor, 10 mM CaCl₂, 100 nM D-Ile-Pro-Arg-AFC diluted in 50 mM HEPES pH 7.5, 150 mM NaCl, 0.1% polyethyleneglycol 8000 and 0.1% bovine serum albumin. A second sample was identical to the first with the addition of factor X inhibitor 10 μM 1-(3-(aminomethyl)phenyl)-N-(3-fluoro-2′-(methylsulfonyl)biphenyl-4-yl)-3-(trifluoromethyl)-1H-pyrazole-5-carboxamide. The assay was carried out in a fluorescence plate reader at room temperature at an excitation wavelength of 400 nm and an emission wavelength of 535 nm. As shown in FIG. 3, the presence of factor X inhibitor 1-(3-(aminomethyl)phenyl)-N-(3-fluoro-2′-(methylsulfonyl)biphenyl-4-yl)-3-(trifluoromethyl)-1H-pyrazole-5-carboxamide (Compound A) reduced the hydrolysis of the factor VIIa substrate D-Ile-Pro-Arg-AFC as compared to the sample without the factor X inhibitor thus demonstrating inhibition of the activation of the coagulation cascade by the factor X inhibitor.

### Example 3

### Assessment of Binding of Factor VIIa Inhibitors to Plasma Proteins

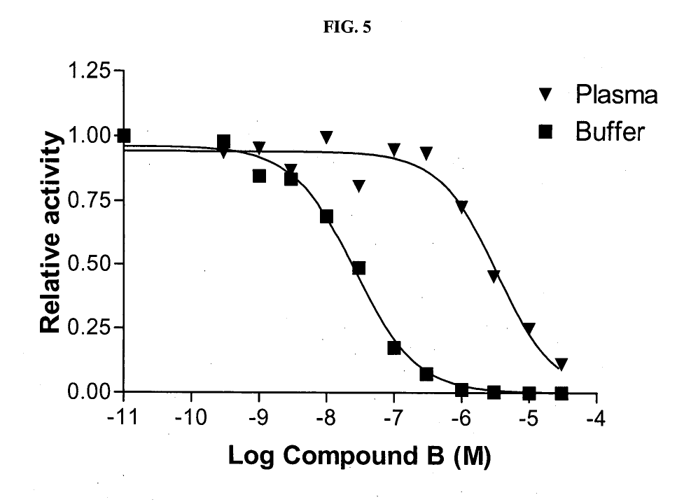

Protein binding of the inhibitor 2-(1-aminoisoquinolin-6-ylamino)-2-(3-ethoxy-4-isopropoxyphenyl)-N-(4-hydroxyphenylsulfonyl)acetamide was determined by measuring factor VIIa inhibition by the test inhibitor in buffer and then in factor X deficient human plasma. As shown in FIG. 4, protein binding of an inhibitor is calculated by determining the shift in inhibitor potency. In FIG. 5, one set of samples contained 2.5 nM factor VIIa, 28 nM soluble tissue factor, 10 mM CaCl₂ 100 nM D-Ile-Pro-Arg-AFC and varying concentrations of factor VIIa inhibitor 2-(1-aminoisoquinolin-6-ylamino)-2-(3-ethoxy-4-isopropoxyphenyl)-N-(4-hydroxyphenylsulfonyl)acetamide (Compound B). The second sample set was identical to the first set with the addition of 90% factor X deficient plasma. All samples were diluted in 50 mM HEPES pH 7.5, 150 mM NaCl, 0.1% polyethyleneglycol 8000 and 0.1% bovine serum albumin. The assay was carried out in a fluorescence plate reader at room temperature at an excitation wavelength of 400 nm and an emission wavelength of 535 nm. The fraction of the inhibitor bound to plasma protein was calculated according to the following formula: 1-IC50 in buffer/IC50 in plasma. As shown in FIG. 5, the IC50 of 2-(1-aminoisoquinolin-6-ylamino)-2-(3-ethoxy-4-isopropoxyphenyl)-N-(4-hydroxyphenylsulfonyl)acetamide in plasma was 3.3 μM and in buffer was 0.026 μM. Protein binding of 2-(1-aminoisoquinolin-6-ylamino)-2-(3-ethoxy-4-isopropoxyphenyl)-N-(4-hydroxyphenylsulfonyl)acetamide was determined to be 99.2%.

### Example 4

### Measurement of Factor VIIa Inhibitor Concentration in Plasma Samples

Plasma was prepared from tested blood samples drawn from rabbits dosed with (R)-N-(5-acetamido-2-(ethylsulfonyl)benzyl)-2-(1-aminoisoquinolin-6-ylamino)-2-(3,4-dimethoxyphenyl)acetamide (Compound C) into buffered citrate. The samples were diluted and combined with tissue factor, factor VIIa, Ca⁺⁺, factor Xa inhibitor 2-(3-Aminomethyl-phenyl)-5-trifluoromethyl-2H-pyrazole-3-carboxylic acid (3-fluoro-2′-methanesulfonyl-biphenyl-4-yl)-amide and fluorogenic factor VIIa substrate D-Ile-Pro-Arg-AFC. The inhibitory activity of the tested sample was measured by monitoring the fluorescence of the D-Ile-Pro-Arg-AFC product and comparing the activity to standard curve constructed with plasma from an undosed rabbit spiked with known concentrations of the (R)-N-(5-acetamido-2-(ethylsulfonyl)benzyl)-2-(1-aminoisoquinolin-6-ylamino)-2-(3,4-dimethoxyphenyl)acetamide. The assay conditions were 10-0.1% rabbit plasma, 2.5 nM factor VIIa, 28 nM soluble tissue factor, 10 mM CaCl₂, 100 nM D-Ile-Pro-Arg-AFC, 10 mM DIN 234944. All samples were diluted in 50 mM HEPES pH 7.5, 150 mM NaCl, 0.1% polyethyleneglycol 8000 and 0.1% bovine serum albumin. The assay was carried out in a fluorescent plate reader at room temperature at an excitation wavelength of 400 nm and an emission wavelength of 535 nm. The concentration of factor VIIa inhibitor in the tested sample is calculated by inhibitor concentration=inhibitor IC50 (as calculated using the standard curve)×plasma dilution factor giving 50% inhibition. IC50 of the standard curve was calculated using floating top and bottom to account for the background signal not originating from factor VIIa activity. The dilution that gave 50% inhibition was calculated fixing the top and bottom based on the values for the uninhibited and fully-inhibited signals obtained from the standard curve with the corresponding plasma dilution (see FIG. 6). The concentration of (R)-N-(5-acetamido-2-(ethylsulfonyl)benzyl)-2-(1-aminoisoquinolin-6-ylamino)-2-(3,4-dimethoxyphenyl)acetamide present in sample B6 was calculated to be 338.5 nM. The concentration was calculated by using the average IC50 for 5 standard curves with plasma concentrations of 10%, 3%, 1%, 0.3%, and 0.1% (data not shown).

In order to assess the accuracy of the disclosed functional assay for measuring factor VIIa inhibitor concentration in plasma samples, the performance of the assay was compared to an LC-MS method. FIG. 7 shows a comparison using 22 samples from rabbits dosed with (R)-N-(5-acetamido-2-(ethylsulfonyl)benzyl)-2-(1-aminoisoquinolin-6-ylamino)-2-(3,4-dimethoxyphenyl)acetamide. The results of this functional assay strongly correlate with concentration of the factor VIIa inhibitor determined by LC-MS, thus validating the accuracy and utility of the functional assay which, unlike the LC-MS method, does not require plasma fractionation.

It will be apparent that certain changes and modifications may be practiced within the scope of the appended claims.

What is claimed is:

1. A method for determining the concentration of a factor VIIa inhibitor in a test plasma sample comprising the steps of:
a) combining the test plasma sample with a solution comprising tissue factor, factor VIIa, a factor Xa inhibitor, a factor VIIa substrate, calcium, and a buffer;
b) determining the concentration of the factor VIIa inhibitor in the test plasma sample by detecting the amount of hydrolysis of the factor VIIa substrate; and
c) comparing the amount of hydrolysis of the factor VIIa substrate in the test plasma sample to an amount of hydrolysis of the factor VIIa substrate in a reference plasma sample having a known concentration of the factor VIIa inhibitor to determine the concentration of factor VIIa inhibitor in the test plasma sample.

2. The method of claim 1 wherein the factor VIIa substrate is selected from the group consisting of D-Ile-Pro-Arg-AFC and H-D-Ile-Pro-Arg-para-nitroaniline.

3. The method of claim 1, wherein the factor Xa inhibitor is selected from the group consisting of 2-(3-Aminomethyl-phenyl)-5-trifluoromethyl-2H-pyrazole-3-carboxylic acid (3-fluoro-2′-methanesulfonyl-biphenyl-4-yl)-amide, tick anticoagulant peptide (TAP), and antistasin.

4. The method of claim 1, wherein the solution comprises tissue factor, factor VIIa, Ca⁺⁺, factor Xa inhibitor 2-(3-Aminomethyl-phenyl)-5-trifluoromethyl-2H-pyrazole-3-carboxylic acid (3-fluoro-2′-methanesulfonyl-biphenyl-4-yl)-amide, and D-Ile-Pro-Arg-AFC.

5. The method of claim 1, wherein the amount of hydrolysis of the factor VIIa substrate is detected by uv or fluorescence spectrophotometry.

6. A method for determining whether a test compound inhibits factor VIIa activity in plasma comprising the steps of:
a) providing a sample comprising factor X deficient plasma, tissue factor, factor VIIa, a factor VIIa substrate, calcium and a buffer;
b) determining the activity of factor VIIa in the sample in the absence of a test compound;
c) determining the activity of factor VIIa in the sample in the presence of the test compound; and
d) comparing the activity of factor VIIa in the presence and absence of the test compound wherein a decrease in activity of factor VIIa in the sample in the presence of the test compound indicates that the test compound inhibits factor VIIa activity.

7. The method of claim 6, wherein the solution comprises tissue factor, factor VIIa, Ca⁺⁺, and D-Ile-Pro-Arg-AFC.

8. The method of claim 6 wherein the factor VIIa substrate is selected from the group consisting of D-Ile-Pro-Arg-AFC and H-D-Ile-Pro-Arg-para-nitroaniline.

9. A method for determining whether a test compound inhibits factor VIIa activity comprising the steps of:
a) providing a sample comprising plasma, a factor Xa inhibitor, tissue factor, factor VIIa, a factor VIIa substrate, calcium and a buffer;
b) determining the activity of factor VIIa in the sample in the absence of a test compound;
c) determining the activity of factor VIIa in the sample in the presence of the test compound; and
d) comparing the activity of factor VIIa in the presence and absence of the test compound wherein a decrease in activity of factor VIIa in the sample in the presence of the test compound indicates that the test compound inhibits factor VIIa activity.

10. The method of claim 9, wherein the solution comprises tissue factor, factor VIIa, Ca⁺⁺, factor Xa inhibitor 2-(3-Aminomethyl-phenyl)-5-trifluoromethyl-2H-pyrazole-3-carboxylic acid (3-fluoro-2′-methanesulfonyl-biphenyl-4-yl)-amide and D-Ile-Pro-Arg-AFC.

11. The method of claim 10 wherein the factor VIIa substrate is selected from the group consisting of D-Ile-Pro-Arg-AFC and H-D-Ile-Pro-Arg-para-nitroaniline.

12. A method for determining the amount of plasma protein binding of a factor VIIa inhibitor comprising the steps of:
a) providing a first solution comprising tissue factor, factor VIIa, a factor VIIa substrate, calcium, and a buffer;
b) providing a second solution comprising a factor X deficient plasma sample, tissue factor, factor VIIa, a factor VIIa substrate, calcium, and a buffer; and
c) determining the amount of plasma protein binding to the factor VIIa inhibitor by comparing the hydrolysis of the factor VIIa substrate in the first solution to the hydrolysis of the factor VIIa substrate in the second solution.

13. The method of claim 12, wherein the factor VIIa substrate is selected from the group consisting of D-Ile-Pro-Arg-AFC, H-D-Ile-Pro-Arg-para-nitroaniline and D-Ile-Pro-Arg-7-amino-4 trifluoromethylcoumarin.

14. A method for determining the amount of plasma protein binding of a factor VIIa inhibitor comprising the steps of:
a) providing a first solution comprising plasma, a factor Xa inhibitor, tissue factor, factor VIIa, a factor VIIa substrate, a factor VIIa inhibitor, calcium, and a buffer;
b) providing a second solution comprising a factor Xa inhibitor, tissue factor, factor VIIa, a factor VIIa substrate, a factor VIIa inhibitor, calcium, and a buffer; and
c) determining the amount of plasma protein binding to the factor VIIa inhibitor by comparing the hydrolysis of the factor VIIa substrate in the first solution to the hydrolysis of the factor VIIa substrate in the second solution.

15. The method of claim 14, wherein the factor VIIa substrate is selected from the group consisting of D-Ile-Pro-Arg-AFC and H-D-Ile-Pro-Arg-para-nitroaniline.

16. A kit for for monitoring factor VIIa inhibitor concentration comprising factor VIIa, a factor VIIa substrate, a factor Xa inhibitor, a reference factor VIIa inhibitor and tissue factor.


SEQUENCE LISTING (1 sequence)

SEQ ID NO: 1 - protein, 207 aa, Homo sapiens
MNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECM